(19) United States
(12) Patent Application Publication
(10) Pub. No.: US 2012/0122075 A1
(43) Pub. Date: May 17, 2012

(54) SYSTEM AND METHOD FOR DETECTING THREATENING AGENTS IN THE AIR

(75) Inventors: Charles J. Call, Albuquerque, NM (US); Ezra Merrill, Albuquerque, NM (US)

(73) Assignee: MesoSystems, Inc., Albuquerque, NM (US)

(21) Appl. No.: 11/627,864

(22) Filed: Jan. 26, 2007

Related U.S. Application Data
(63) Continuation-in-part of application No. 11/558,269, filed on Nov. 9, 2006.
Application No. 11/558,269 is a continuation-in-part of application No. 11/058,442, filed on Feb. 15, 2005, now abandoned.
Application No. 11/058,442 is a continuation-in-part of application No. 10/066,404, filed on Feb. 1, 2002, now Pat. No. 6,887,710.
Application No. 10/066,404 is a continuation-in-part of application No. 09/775,872, filed on Feb. 1, 2001, now Pat. No. 6,729,196.
Application No. 09/775,872 is a continuation-in-part of application No. 09/265,619, filed on Mar. 10, 1999, now Pat. No. 6,267,016.
Application No. 09/265,619 is a continuation-in-part of application No. 09/265,620, filed on Mar. 10, 1999, now Pat. No. 6,363,800.
Application No. 10/066,404 is a continuation-in-part of application No. 09/955,481, filed on Sep. 17, 2001, now Pat. No. 6,695,146.
Application No. 09/955,481 is a continuation-in-part of application No. 09/191,980, filed on Nov. 13, 1998, now Pat. No. 6,062,392.
Application No. 09/191,980 is a continuation-in-part of application No. 09/494,962, filed on Jan. 31, 2000, now Pat. No. 6,290,065.
(60) Provisional application No. 60/337,674, filed on Nov. 13, 2001.

(51) Int. Cl. C12Q 3/00; C12M 1/36; F24F 11/04; G01N 21/75; B01D 46/46; A61L 2/24; G01N 33/53

(52) U.S. Cl. 435/3; 422/3; 422/62; 435/286.1; 436/501; 436/50; 95/1; 454/256; 454/255

(57) ABSTRACT

A multi-tier approach for use in a detecting harmful agents conveyed by the air. In a first tier procedure, the air (in a structure or a predefined area) is continuously automatically screened at a plurality of different predefined locations by air sensors distributed in the area to be monitored. Each air sensor is configured to detect a potentially harmful substance that is carried by the air proximate the predefined location, to determine if a potentially harmful substance might be present, but need not identify a specific harmful substance. When a potentially harmful agent is identified by an air sensor in the first tier screening, a sample of the potential threat is collected, and a second tier procedure is initiated. The second tier procedure uses a manual test, such as a nucleic acid amplification and detection assay to detect any of a plurality of different specific threats in the sample.

### RELATED APPLICATIONS

This application is a continuation-in-part of prior copending U.S. patent application Ser No. 11/558,269, filed on Nov. 9, 2006, which itself is a continuation-in-part of prior copending U.S. patent application Ser. No. 11/058,442, filed on Feb. 15, 2005, which itself is a continuation-in-part of a prior U.S. patent application Ser. No. 10/066,404, filed on Feb. 1, 2002, which issued as U.S. Pat. No. 6,887,710 on May 3, 2005, and which itself is based on prior U.S. Provisional Patent Application Ser. No. 60/337,674, filed on Nov. 13, 2001, the benefits of the filing dates of which are hereby claimed under 35 U.S.C. §119(e) and 35 U.S.C. §120. U.S. patent application Ser. No. 10/066,404 is a continuation-in-part of prior U.S. patent application Ser. No. 09/775,872, filed on Feb. 1, 2001, which issued as U.S. Pat. No. 6,729,196 on May 4, 2004 and which is itself is a continuation-in-part of U.S. patent application Ser. No. 09/265,619, filed on Mar. 10, 1999, which issued as U.S. Pat. No. 6,267,016 on Jul. 31, 2001, and of prior U.S. patent application Ser. No. 09/265,620, filed on Mar. 10, 1999, which issued as U.S. Pat. No. 6,363,800 on Apr. 2, 2002, the benefit of the filing dates of which are hereby claimed under 35 U.S.C. §120. Further, U.S. patent application Ser. No. 10/066,404, is also a continuation-in-part of prior U.S. patent application Ser. No. 09/955,481, filed on Sep. 17, 2001, which issued as U.S. Pat. No. 6,695,146 on Feb. 24, 2004 and which itself is a continuation-in-part of prior U.S. patent application Ser. No. 09/191,980, filed on Nov. 13, 1998, which issued as U.S. Pat. No. 6,062,392 on May 16, 2000, and of U.S. patent application Ser. No. 09/494,962, filed on Jan. 31, 2000, which issued as U.S. Pat. No. 6,290,065 on Sep. 18, 2001, the benefit of the filing dates of which is hereby claimed under 35 U.S.C. §120.

### BACKGROUND

In 2001, a small volume of Bacillus anthracis (anthrax) entered the American Media Building in Florida, likely via mail delivered to the building. The contamination spread throughout the 70,000 square foot office building, resulting in one fatality and the abandonment of the building for a period of years. The building was later sold for $40,000, a fraction of its actual worth, and decontamination costs required to place the building back into service are expected to range from $10-100 million.

In response to the threat posed by intentionally contaminated mail, most of the incoming mail passing through the larger United States Postal Service (USPS) mail distribution centers is now screened for anthrax. However, not all mail handled by the USPS passes through one of these distribution centers. Furthermore, the USPS mail screening system only detects Bacillus anthracis (i.e., anthrax), but currently does not attempt to detect ricin, tularemia or any of the other biological hazardous threats or “bio-threats.” Also, bulk mail such as boxes of pamphlets, and mass advertising mailings are not screened by the USPS, and are often shipped by overnight carriers such as United Parcel Service (UPS), which does not screen any packages for bio-threats.

Furthermore, while the mail does present a likely route by which a harmful substance can be introduced into a building, it is by no means the only possible delivery mechanism. It is recognized that a harmful substance can be brought into the building and then be introduced into the air of the building, to be distributed throughout the building by the building's ventilation system. A harmful substance can similarly be introduced into the air outside the building, and the contaminated air can then be drawn into the building through ventilation intakes, doors, and windows.

Buildings are not the only areas that can be threatened by airborne agents. Other potential targets at risk include stadiums (both indoor and outdoor), transportation facilities (airports, train stations, bus depots, ports, etc.), educational facilities, entertainment facilities, military facilities, governmental facilities, and vehicles (aircraft, trains, buses, etc.).

Air monitoring systems are available to detect such threats, but currently available systems are generally too expensive for widespread deployment. A key issue is that analytical components sufficiently sophisticated to specifically identify a threatening agent are very expensive, making the task of monitoring relatively large facilities, such as large buildings, stadiums, or airports, prohibitively expensive. It would thus be desirable to provide a less costly air monitoring system, which can make the use of such air monitoring systems as common as smoke alarm systems.

### SUMMARY

Accordingly, an approach has been developed for screening air for contamination in a multi-tier approach. In general, the approach relies on distributing a plurality of air sensors over an area to be monitored. Significantly, the plurality of air sensors are configured to broadly determine if a potentially harmful substance might be present, as opposed to identifying a specific harmful substance. As such, the air sensors can be relatively inexpensive, enabling the air sensors to be widely deployed in a sensor network. For use in a building, such air sensors can be deployed on every floor, or in every publicly accessible location, or in each different business or agency occupying the building. For an airport, the air sensors might be deployed, for example, at each ticket counter, at each security checkpoint, at each gate, and at each baggage carousel. It should be recognized that these proposed sites for sensor deployment are intended to be exemplary, rather than limiting. The artisan of ordinary skill will recognize that many different deployment configurations are possible.

Each of the plurality of air sensors is configured to automatically screen the air proximate to the air sensor for contaminants (again noting that such screening is intended to determine if a potentially harmful substance might be present, as opposed to identifying a specific harmful substance). Once a specific air sensor determines that a potentially harmful substance might be present, a sample of the potentially harmful substance is collected, and an analytical device is used to verify whether a harmful substance is actually present (and preferably, also identify the harmful substance). If the analytical device is portable, it can be brought to the area in which the potentially harmful substance was detected, or the sample can be taken to the analytical device. The total cost of ownership for this approach is modest, because the plurality of air sensors can be implemented using relatively inexpensive technology and thus, these sensors can be widely deployed. While the verifying analytical device is relatively more sophisticated and expensive, a single such analytical device can support a large number of air sensors. The plurality of air sensors can be configured to operate automatically over an extended period of time, so that the manpower requirements for implementing this approach are minimal. To deploy a sensor network, individual air sensors are preferably linked to a controller, such that the controller can identify a specific air sensor that has potentially detected an airborne threat, so that response personnel can be dispatched to the specific air sensor to retrieve the sample, and either perform the analysis proximate the air sensor, or take the sample to a different location for analysis. The use of a portable analytical verification device will reduce the system response time (i.e., the time between the identification of a potential airborne threat and the verification that an actual airborne threat has been detected), because the sample can be analyzed immediately, without wasting time transporting the sample to a remote analytical lab.

Different air sensors can be optimized to detect a particular airborne threat. In one exemplary embodiment, the plurality of air sensors are optimized to screen for potentially harmful biological particles. In another exemplary embodiment, the plurality of air sensors are optimized to screen for potentially harmful radiological particles, while in yet another exemplary embodiment, the plurality of air sensors are optimized to screen for potentially harmful chemical agents. As described in greater detail below, a particularly useful air sensor can be configured to detect biological threats by measuring fluorescent properties of particles collected from the air that is proximate to the air sensor. Gamma ray detectors and/or Geiger counters can be incorporated into air sensors to screen for radioactive particles. Relatively inexpensive metal oxide sensors, which can detect specific classes of chemicals, can be incorporated into air sensors to screen for chemical contaminants If desired, different types of air sensors can be deployed in different areas, and air sensors can be implemented including more than one type of sensor. Significantly, such exemplary air sensors respond relatively quickly to the presence of contaminants in air that is proximate to the air sensors. As a result, a little time is required between the release or introduction of a contaminant into the air proximate to the air sensor and the detection of the contaminant by the sensor. In a particularly preferred embodiment, the air sensors are configured to operate continuously and require minimal consumables, such that extended maintenance-free deployment is achieved (further reducing manpower requirements for service and maintenance, and further reducing the total cost of ownership).

In an exemplary embodiment, each air sensor is configured to communicate with the sensor network or a network controller, so that each sensor can indicate to the network that a potentially harmful substance has been detected. The indication will identify the location of the air sensor that detected the potentially harmful substance, so that personnel can be dispatched to that location to obtain the sample so that it can be analyzed by the analytical device, which is configured to confirm the indication (or to specifically identify the harmful substance, or both). The plurality of air sensors can be configured to automatically collect a sample for verification once a potentially harmful substance has been identified, or the personnel dispatched to the location of the air sensor detecting the potential contaminant can be tasked with collecting the sample for verification. In an exemplary working embodiment of such a networkable air sensor, the network connection is established by electrically coupling the air sensor to a controller. Thus, a plurality of such air sensors can be coupled together to form a network, with one or more computers being used to control the network. It should be recognized that other types of network connections are possible (i.e., connections other than hardwired electrical connections), including but not limited to wireless connections, such as infrared and radiofrequency connections (or any combination thereof). While general purpose computers represent one example of a suitable network controller, it should be recognized that application specific integrated circuits (ASICs), custom computing devices, and hardware based network controllers are encompassed within the spirit of the concepts disclosed herein.

As used herein and in the claims that follow, the term “airborne threat” is intended to encompass a hazardous biological agent or bio-terror threat or bio-warfare threat, including any living organism (e.g., virus, bacteria, bacterial spore, or fungus) that is pathogenic (disease causing), any toxin that may be extracted from or produced by an organism (i.e., a plant, an animal, or fungus), as well as chemical and radiological agents that can harm people or property. In some embodiments, such airborne threats are assumed to encompass particles of respirable size, that is, particles ranging from about 1 to about 10 microns in aerodynamic diameter.

An exemplary method for carrying out this approach begins with a first tier screening of the air. During the first tier screening, the air to be monitored (which can be air within a man made structure, or air that is proximate to a defined geospatial area, such as an open sports stadium or a park) is screened by a plurality of air sensors distributed in the area (or structure) to be monitored, to detect a potential threat that may be conveyed by the air. If a potential airborne threat is detected during the first tier screening by one or more of the distributed air sensors, a sample of the potential airborne threat is collected proximate to the air sensor that detected the potential airborne threat, and a first tier alarm is produced, indicating that a potential airborne threat has been detected. Next, a second tier screening of the sample collected during the first tier screening is carried out to attempt to identify a specific type of airborne threat comprising the sample. If it is confirmed by the second tier screening that the sample comprises a specific airborne threat (or the second tier screening confirms that an airborne threat is present, without specifically identifying the agent), a series of predefined appropriate steps can be initiated to limit contamination by preventing the specific airborne threat from spreading beyond the air sensor that detected the airborne threat, to limit exposure of personnel to the specific airborne threat that has been identified or verified in the second tier analysis.

The method can further include the step of periodically carrying out a third tier screening to detect a potential airborne threat in at least one additional sample. This additional sample can either be a background sample collected over time from air circulated within the area to be monitored, or can be a background sample collected over time at one or more specific locations in the area to be monitored.

Preferably the plurality of air sensors are configured to continuously monitor the air. In an embodiment optimized for the detection of biological threats, the step of continuously screening the air using a plurality of air sensors during the first tier screening can include the following steps. These steps can be implemented by each air sensor, although it should be recognized that some systems will include a mix of sensors optimized to detect different types of airborne threats, such as biological, chemical, and radiological threats, and in some systems, fewer than all sensors will implement the following steps to detect bio-threats. The steps include collecting airborne particles proximate to the air sensor, and then irradiating the particles that are collected with light of a predefined waveband. A fluorescence signature of light emitted from the particles when thus irradiated is detected. Based upon the fluorescence signature, the method provides for automatically determining if the particles comprise a potential bio-threat. The step of continuously screening the air during the first tier can also include the steps of collecting the particles conveyed by the air proximate to the air sensor, and then impacting the particles onto a surface. The surface can be a solid surface, which allows for a bulk measurement of the fluorescence properties, or can be a gel that contains biological molecules, such as stains or dyes or labeled antibodies that bond with bio-threat particles. One or more biological molecules are selected a priori such that bio-threat particles are easily detected, for example, by fluorescence detection. The step of continuously screening the air proximate to the air sensor in the first tier can also include mixing a secondary aerosol with the particles conveyed by the air. The secondary aerosol contains the biological molecules to be used to detect the bio-threats, such that the bio-threat particles are more easily detected.

For systems optimized to detect bio-threats, the second tier screening can include performing a polymerase chain reaction (PCR) assay test of the sample that was collected during the first tier screening, wherein the PCR assay test is configured to identify at least one specific bio-threat. Alternatively, or in addition, the second tier screening can include the step of performing an immunoassay test on the sample that was collected during the first tier screening. The immunoassay test is selected to identify at least one specific bio-threat. The second tier screening may be split into two steps, wherein the first step is to perform a test that is different than the first tier screening, so as to confirm whether the possible bio-threat indeed has at least one additional characteristic indicative of an actual bio-threat, and if so, then performing an analysis that attempts to identify the specific type of bio-threat comprising the sample. It should be recognized that PCR is but one of many different types of nucleic acid amplification and detection assays available, and as such, PCR is intended to be exemplary, rather than limiting. Other suitable nucleic acid amplification and detection assays can also be employed.

Another aspect of the approach discussed herein is directed to an exemplary system configured for use to monitor the air in a predefined area using a distributed network of air sensors, each air sensor being configured to broadly determine if a potential airborne threat might be present, as opposed to identifying a specific airborne threat. The exemplary system also includes a verification analytical device that can be used to analyze a sample collected whenever one of the distributed air sensors detects a potential airborne threat, to verify whether an actual threat is present. The exemplary system uses components that carry out functions generally as described in regard to the exemplary method discussed above.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWING

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1A is a block diagram of an exemplary sensor network in which the approach described below is implemented;

FIG. 1B is a block diagram of another exemplary sensor network in which the approach described below is implemented in a structure;

FIG. 2 is an elevational view of an exemplary automatic air sensor and sampler device for detecting potential airborne threats in real time, which can be employed as a first tier detector;

FIG. 3 is a plan view of an exemplary second tier detector, which in this example is a PCR type detector useful for providing positive confirmation of any bio-threat detected by the automatic air sensor/sampler device of FIG. 2;

FIG. 4 is a schematic block diagram illustrating an exemplary procedure used to continuously screen air to detect possible bio-threats; and

FIG. 5 is an exemplary schematic illustration of the process flow employed in one exemplary embodiment of the present approach.

### DESCRIPTION

### Figures and Disclosed Embodiments Are Not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein. In particular, portions of the disclosure that follows specifically describe the detection of airborne biological threats. It must be recognized that the concepts disclosed herein are equally applicable to detecting airborne chemical and radiological threats using a two tier approach (or in cases where background samples are collected, a three tier approach).

### Air-Screening to Detect Potential Airborne Threats

A key motivation for employing the USPS solution discussed above under the Background section is that the USPS system has a very low false alarm rate (<1 false alarm/year, although the initial deployment of the USPS system only scans for anthrax) and a very high probability that anthrax powder (spores) will be detected by the USPS system if present in the mail being screened.

The following describes an alternative novel system that also should achieve these goals (i.e., both a very low false alarm rate and a very high probability of detecting airborne threats), but which is more practical and affordable, and which can be deployed to screen air in many different locations. As described in greater detail below, an exemplary embodiment is optimized to detect bio-threats. It should be recognized however, that a sensor network as described herein can be configured to screen air for the presence of chemical threats and radiological threats as well. If desired, such a sensor network can include different types of sensors, to detect different types of airborne threats. The sensor network described herein is based on deploying a plurality of air sensors to screen the air at different locations distributed throughout an area to be monitored. The plurality of sensors are not configured to positively identify an airborne threat; instead, the sensors are expected to identify potential airborne threats, such that additional testing is required to positively identify an airborne threat (be it biological, chemical, or radiological in nature) or verify that an airborne threat is indeed present. The sensor network (i.e., the plurality of distributed air sensors communicating with one or more controllers) represents a first tier, and the resources required to verify the presence of an airborne threat and/or positively identify the airborne threat represent a second tier. Significantly, the sensors employed in the first tier are relatively inexpensive, and once deployed require a relatively small amount of man-hours to maintain. In contrast, the resources required to implement the second tier are much more capital and man-power intensive; however, the resources required to implement the second tier can support a relatively large sensor network, such that the total cost (the combination of the first tier and second tier costs) to implement a sensor network for monitoring the air in a relatively large area is favorable when compared to alternative technologies, which rely on employing a network of relatively expensive detectors (each individual sensor often being as capital intensive as the entire second tier required to support the sensor network disclosed herein). Thus, an advantage of the sensor network described herein is that capital intensive resources in the second tier can be leveraged to enable the monitoring of a larger area than can be monitored by a conventional capital intensive detector.

Many embodiments of such a multi-tier sensor network are possible. In at least one embodiment, the widely distributed first tier sensors are configured to automatically collect a sample of a potential threat for second tier testing, whenever the first tier sensor detects a potential threat. In at least one other embodiment, a sampler configured to collect a sample for second tier testing is co-located with the first tier sensors, such that the co-located sampler obtains the second tier sample (i.e., the first tier sensor itself does not collect a sample for second tier testing; the co-located sampler performs that function). In yet another embodiment, response personnel are dispatched to the first tier sensor detecting a potential threat to collect the second tier sample. Regardless of how the sample is collected, preferably trained response personnel are tasked with transporting the second tier sample to the second tier analytical device for verification/identification of the threat agent.

### Exemplary Multi-Tier Airborne Threat Detection System

Providing a cost effective sensor network to detect airborne threats requires innovative use of hardware appropriate to accomplish the desired goals, combined with a solid concept of operations (CONOPS). In order to reduce cost and minimize contamination of an area being monitored by the sensor network described herein, a “detect-to-protect” system is needed that provides a near-real-time detection with, for example, a 1-2 minute response time (recognizing that such a time period is intended to be exemplary, and not limiting). This continuous, near-real-time goal can minimize the spread of airborne threats within an area being monitored (where control of air flow in the area can be implemented), and the exposure of personnel to contamination by such airborne threats. Costs of such a system can be reduced by carrying out tests that consume assays only after a near-real-time warning sensor has produced an alarm, indicating that an airborne threat may be present, and additionally, at the end of each day (or after some other extended period of time), when checking for background levels of airborne threats at one or more locations in the area.

An exemplary system for detecting airborne threats (including biological, chemical or radiological threats), should have the following characteristics when the full ensemble of components comprising the system are deployed:

Automatically screens air with integrated rapid detection of potential threats;

Provides an immediate warning to security or other designated personnel when a potential threat has been identified, such a warning specifying the sensor/location where the potential threat has been detected;

Automatically collects a sample of the potential threat (alternatively, the sample can be collected by response personnel, although such an embodiment will likely increase response time); and

Implements rapid on-site or nearby analysis of the sample of the potential threat to verify that a threat is present, and/or to positively identify the threat.

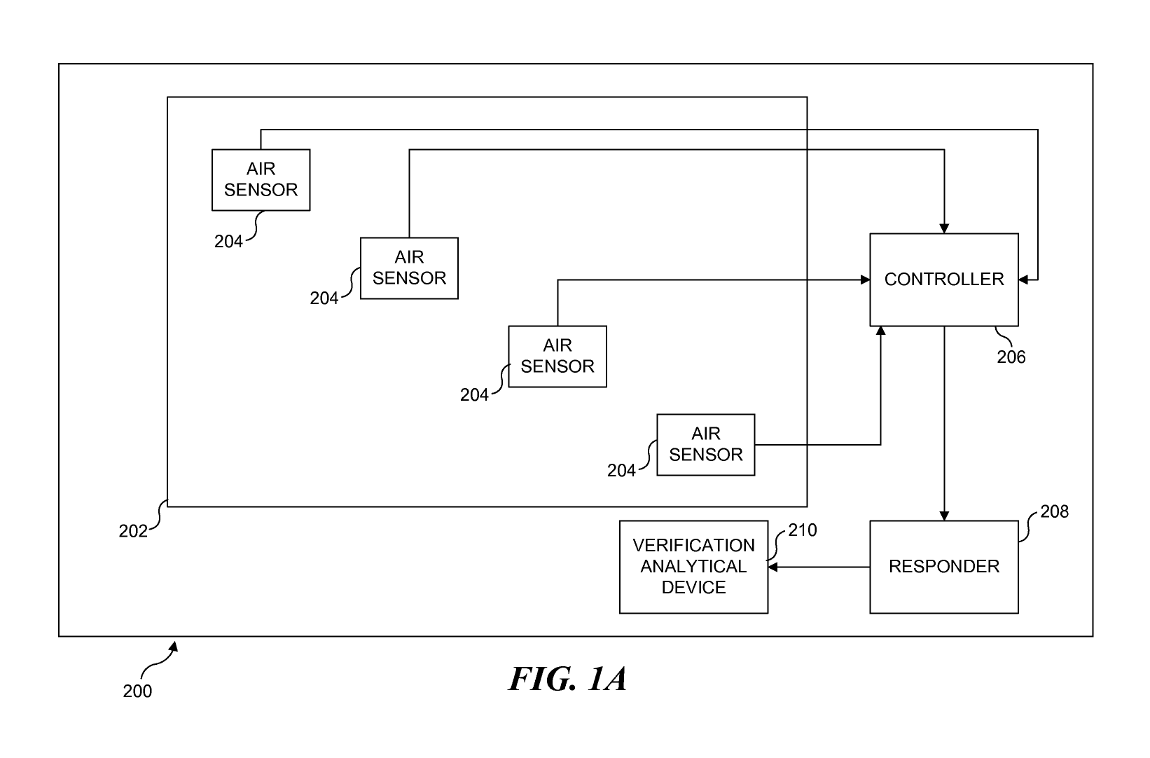

An exemplary sensor network 200 is schematically illustrated in FIG. 1A and includes a plurality of air sensors 204 deployed to monitor the air in an area 202. Significantly, each air sensor 204 is not intended to specifically identify an airborne threat, but instead, is intended to detect potential threats. Each air sensor will include one or more components configured to detect (and in some, but not all embodiments, possibly classify) biological, chemical, or radiological threats, but not to identify a specific harmful substance. Thus, the air sensors individually can be implemented relatively inexpensively, enabling the air in a relatively large area to be screened at a relatively modest cost. The specific spatial distribution of the plurality of air sensors can be modified as desired. In general, the air sensors will exhibit a range over which they are most effective. In one exemplary distribution, the air sensors are distributed such that substantially the entire area to be monitored is within the effective range of at least one air sensor. In another exemplary distribution, the air sensors are distributed such that air sensors are concentrated in key areas, rather than being distributed to monitor the entire area. In a sports stadium, such keys areas are likely to include entrances, exits, and seating areas. In an aircraft, the passenger cabin might be considered to be a key area, while the cargo area is considered less critical. In a building, areas accessible by the general public and intakes for the building's HVAC system are likely to be considered to be key areas. Of course, it should be understood that such distributions and the identifications of key and less critical locations are intended to be exemplary and not limiting. Those of ordinary skill in the art will readily recognize that many different air sensor distributions are possible, and that the air sensor distribution implemented will often be a function of characteristics of the area being monitored. For example, a risk analysis can be performed to identify threats and vulnerabilities, and rank the identify the areas of greatest risk to an attack with chemical, biological or radiological materials.

Each air sensor also includes a communication interface configured to enable the air sensor to communicate with a controller 206. Note that controller 206 can be disposed in area 202 that is being monitored, or outside of the area (as shown in FIG. 1A). In an exemplary embodiment, each air sensor is hardwired to the controller; however, those of ordinary skill in the art will readily recognize that many types of data links can be implemented, including, but not limited to, the use of universal serial bus (USB) ports, parallel ports, serial ports, FireWire ports, infrared data ports, and wireless data communication such as Wi-Fi and Bluetooth™, network connections via Ethernet ports, and other connections that employ the Internet or other types of networks.

When one of the plurality of air sensors detects a potential airborne threat, that air sensor sends a signal to the controller indicating that a potential airborne threat has been detected. The controller will in turn alert a responder 208 (one or more individuals tasked with responding to the detection of a potential threat) that a potential threat has been detected, and the controller will also provide the responder with the location of the specific air sensor that detected the potential airborne threat.

The responder is dispatched to the specific air sensor that detected the potential airborne threat. In one embodiment, the responder is tasked with collecting a sample of the potential airborne threat proximate to the specific air sensor that signaled the controller. In a particularly preferred embodiment, whenever an air sensor sends an alert signal to the controller indicating that a potential airborne threat has been detected, the air sensor automatically collects a sample of the potential airborne threat, such that the responder need only retrieve the sample collected by the air sensor. In some embodiments, the sample comprises particles collected from the air, whereas in other embodiments the sample comprises a volume of air. Generally, biological and radiological threats comprises particles, while chemical threats can comprise both particles and vapors (i.e., gases). The responder is then tasked with ensuring that the sample of the potential airborne threat detected by the air sensor is analyzed using verification analytical device 210.

Verification analytical device 210 is configured to either specifically identify the potential airborne threat, or to determine if the potential threat is real, so that appropriate action required to protect personnel and property can then be taken. In general, verification analytical device 210 is implemented by relatively sophisticated and expensive analytical equipment. Where the potential airborne threat represents a radiological threat, each individual air sensor (generally a gamma ray detector, although it should be recognized that other radiation detectors, configured to respond to alpha radiation and/or beta radiation, can also be employed) will likely be configured to simply detect the presence of a radiological particle or an abnormally high level of radiation in an aerosol, without being able to determine what radiological material is present, while verification analytical device 210 will be configured to specifically identify what radiological isotope is present. Where the potential airborne threat represents a chemical threat, each individual air sensor will likely be configured to simply detect the presence of a class of chemical threats, without specifically identifying the chemical species, while verification analytical device 210 will be configured to specifically identify the chemical. For example, a combination of a gas chromatograph and a mass spectrometer (i.e., a GCMS) represents a particularly useful verification analytical device 210 to identify airborne chemical threats. Note that GCMS devices are typically expensive, and are thus not suited to be widely deployed as air sensors, but can be beneficially employed as a single verification analytical device 210 supporting a plurality of relatively less sophisticated and less expensive air sensors. Examples of low-cost chemical sensors (for incorporation into the individual air sensors) include thin-film metal oxide sensors and surface acoustic wave sensors, although such technologies are intended to be exemplary, rather than limiting. Where the potential airborne threat represents a biological threat, each individual and air sensor will likely be configured to simply detect the presence of a biological agent, without specifically identifying the biological agent, while verification analytical device 210 will be configured to specifically identify the biological agent. An exemplary verification analytical device 210 particularly well suited to identify biological agents is described in greater detail below.

The time that elapses between the detection of a potential airborne threat by one of the plurality of air sensors and verification/identification of the airborne threat by the verification analytical device is important. Clearly, it would be desirable to minimize the time between the initial detection of a potential airborne threat and the verification that an actual airborne threat is present. Thus, in particularly preferred embodiments of the sensor networks described herein, the verification analytical device can provide either verification that an actual threat is present (or identify a specific threat agent) relatively quickly (e.g., in less than about 30 minutes, although it should be recognized that such a time period is intended to be exemplary, rather than limiting). Several sensor network parameters can be manipulated to further reduce the required response time. For example, response personnel should be on call, such that they can be dispatched without delay to the specific sensor detecting the potential threat. Air sensors that are configured to collect the sample, rather than requiring the responder to collect the sample, will further reduce the response time (i.e., the time elapsing between the initial detection of a potential airborne threat and the verification that an actual airborne threat is present), because the sample will be waiting for the responder when they arrive at the air sensor that initially detected the potential threat. Providing a portable verification analytical device can also reduce the response time, because the verification analytical device can be taken by the responder to the air sensor that detected the potential threat, so that the verification analysis can be performed proximate to the air sensor detecting the potential threat (eliminating the time required to transport the sample of the potential threat to a verification analytical device disposed elsewhere). Portable verification analytical devices that are not sufficiently small to be carried by a person can be incorporated into a service cart that can be moved to the air sensor that initially detected the potential threat, or can be incorporated into a vehicle that can be driven as close as practical to the air sensor that initially detected the potential threat.

Once the verification analytical device has determined that an actual threat is present (or has specifically identified the threat agent), appropriate responses can be implemented to reduce the danger to people and property. Emergency response personnel can be called to the scene. Personnel near the air sensor detecting the threat can be evacuated and treated. Where possible, the area proximate to the air sensor detecting the threat can be isolated from other areas (for example, if the air sensor is in a room in a building, the HVAC system of the building can be manipulated to prevent air in that room from being distributed throughout the rest of the building). Those of ordinary skill in the art will readily recognize that the appropriate response will likely be a function of the specific threat detected and the area being threatened. A response plan for each building or facility should specify what actions are taken in response to specific threats. It should be understood in this context that other building components that affect ventilation, such as doors, windows and elevators, may be considered to be components of the HVAC system.

FIG. 1B is a block diagram schematically illustrating a sensor network 10 configured to be deployed in a building or other structure. A plurality of air sensors 24 are distributed in a structure 12. Each air sensor 24 is logically coupled to a controller 28. In the exemplary system shown in FIG. 1B, a MesoSystems Technology, Inc. AirSentinel® monitor is employed as each air sensor 24. However, it will be understood that other types of continuous monitors that can detect a potential airborne threat might instead be used in the present system. Preferably, each air sensor operates automatically, continuously, and is capable of detecting a potential threat in near real-time.

If an alarm signal is generated by the near-real-time air sensor 24, e.g., by an AirSentinel® monitor, then a signal is transmitted either by a wire or wireless communication signal to controller 28. In addition, a second sample is collected by the near-real-time detector (or a separate sampler is triggered by said detector) for analysis in a verification analytical device (which in one exemplary embodiment is a PCR-based agent identification system configured to detect biological agents) in a second tier of this approach. Controller 28 can be a conventional personal computer, a hardwired logic device, an ASIC, or some other computing device or logic device that is configured to carry out specific functions as discussed herein. Controller 28 can respond to the detection of a potential airborne threat by causing an audible or visual alarm 32 to be activated, and to send a page message or other type of message by wire or wirelessly, to initiate a second tier response (autonomously if such equipment is installed, or by summoning trained personnel such as the responder of FIG. 1A), to carry out further testing of the sample that was collected by the near-real-time detector, e.g., by the AirSentinel® monitor. This second tier testing can be done manually with a device at a facility 30, which can be in structure 12 (or at a different location) to confirm whether an airborne threat has actually been detected and if so, to identify a specific threat agent included in the sample.

In an exemplary (but not limiting) embodiment configured to detect airborne biological threats, the device used for this second tier determination is a portable device, such as Idaho Technology Inc.'s Razor™ or Cepheid, Inc.'s GeneXpert bio-agent identification systems, both of which employ PCR technology to identify a number of different specific bio-threat agents based upon the DNA of such samples, providing results in about 20-30 minutes. Immunoassay or microbial or protein stain tests can also be used to test for specific bio-threat agents or specific classes of bio-threats, such as anthrax, ricin and botulinum toxin. A portion of the sample can be sent to a laboratory for formal confirmation of any specific bio-threat agent identified or to confirm the absence of such a bio-threat. As discussed in detail above, other types of verification analytical devices can be employed, such as verification analytical devices configured to identify chemical threats, and verification analytical devices configured to identify radiological threats.

Each air sensor 24 included in system 10 is also fitted with a continuous background air sampler 26 that continuously collects particulates from the air being screened at the air sensor. At the end of the day, or at some other predefined period interval of time, these background air samples can be tested to determine whether a lower concentration of an airborne threat has been collected over time at the air sensor where the background air sampler was installed. The detection of a background air filter will not result in an immediate alarm, but serves as a third tier of detection to minimize the risk that lower concentrations of an airborne threat being carried by the air will not be detected by the near-real-time detection system. An exemplary background air sampler is a dry filter sampler, such as those manufactured by Murtech, Inc.

It is also important to detect background levels of an airborne threat that may be dispersed within structure 12 being protected by system 10. To temperature condition the air within structure 12, a heating ventilation air conditioning (HVAC) system 38 draws structure air through one or more room air intakes 34. Any potential airborne threat particles that have been picked up and carried by the air are collected over time on a background air sampler 36 before being drawn into the HVAC evaporator or heating coil temperature conditioning components and exhausted back into the structure as temperature-conditioned return air. At predefined time intervals, such as at the end of each work shift in structure 12, background air sampler 36 can be extracted and particulates collected can be checked to identify any potential airborne threats comprising the particles filtered from the structure air. This check is another part of the third tier of detection of an airborne threat attack being promulgated via air passing through structure 12. If a potential airborne threat is detected in the background sample removed from background air sampler 26 at air sensors 24, or in the background sample removed from the air handling system for structure 12, (i.e., background air sampler 36), the detection will be confirmed and if so, the specific airborne threat can be identified by summoning the trained personnel to implement the second tier evaluation of the background sample. Once again, an exemplary background air sampler is a dry filter sampler, such as those manufactured by Murtech, Inc.

A sensor network as described herein thus includes at least two tiers—the plurality of air sensors (the first tier), and the verification analytical device (the second tier). A third tier (the background sampler) can be added to detect threat agents present in levels that are too low to be detected by the first tier. These three layers, or tiers, of processing and technology provide a level of redundancy, achieving both a continuous monitoring on the first tier, and confirming any potential airborne threat that is detected in a timely manner in the second tier. A “detect to warn” rapid threat capability is backed up with a “detect to treat” capability, which is also implemented in regard to the third tier after evaluating samples taken over a period of time. This approach leads to a system that has a high probability of detecting large events in near-real-time, but is still able to provide delayed detection of low-level threats.

### Bio-Detection Technology

As noted above, a particularly useful sensor network will be configured to detect biological threats, in order to protect areas from biological contamination such as anthrax. The table below highlights and summarizes the technologies and objectives of each tier in a multi-tier sensor network configured to screen air for biological threats.

| Tier | Technological Approaches | Objectives |
|---|---|---|
| 1 | Ultraviolet light-induced | Discriminate bio-threat particles from paper |
| | fluorescence (UV-LIF), | dust, corn starch, and other non-threat |
| | including fluorescence enhanced | particles |
| | with microbial, nucleic acid or | |
| | protein stains | |
| | Particle counts/size/shape | Additional information to support decision on |
| | | threat vs. non-threat particle clouds |
| | Rotating impactor air sampler or | Collects a sample when the UV-LIF or |
| | cyclonic air sampler, or | particle counter detects a possible threat event. |
| | impinger air sampler | |
| | (Infrared) IR or RAMAN | Specifically identify non-biological powders |
| | Spectrometer | such as starch, Equal ™ sweetener and talc, |
| | | indicates when a bio-threat threat may be |
| | | present. |
| 2 | Nucleic acid amplication and | Specifically identifies bio-threat agents. |
| | detection (e.g., PCR) | |
| | Immunoassay tests | Test for specific toxins such as ricin and |
| | | botulinum toxin and as an alternative to |
| | | nucleic acid assays |
| | Microbial, nucleic acid or | Confirm if unknown sample is of biological |
| | protein stains (to be used in | origin |
| | conjunction with other Tier 2 | |
| | tests) | |
| 3 | Background air sampler (e.g., | Continuously collects a background sample |
| | dry filter sampler) | from mailroom and/or from each mail |
| | | processing station during all mailroom |
| | | operations to enable detection of small |
| | | releases not detected by Tier 1 alarms |
| | Nucleic acid amplication and | Tests background air samples for bio-threat |
| | detection (e.g., PCR) | agents. |

### Tier 1 Alarm Technology

As noted above, in one exemplary embodiment, the first tier of detection uses AirSentinel® monitor as air sensor 204 (FIG. 1A) or air sensor 24 (FIG. 1B). Each AirSentinel™ monitor contains both a particle counter/sizer and a sensor based on ultraviolet-light-induced fluorescence (UV-LIF). The AirSentinel® monitor for continuously monitoring indoor air is readily adapted for use in the sensor networks described herein and can be easily fitted to many types of support structures or surfaces. However, other types of continuous monitoring devices might instead be used for the near-real-time detector employed for screening air in the first tier of the sensor networks disclosed herein.

Fluorescence from biological materials is generally distinct from that of most other materials, and in particular, from corn starch and road dust (i.e., dirt). For example, the fluorescence is yellow-green from Bacillus spores (including anthrax spores) and bluish-red from corn starch, when illuminated with 365 nm ultraviolet light. An equivalent mass of road dust does not emit significant fluorescence when illuminated with 365 nm light. The AirSentinel® monitor incorporates color filters on the photo detectors included within it, to enable it to distinguish bio-aerosols from corn starch aerosol. However, paper dust often contains a high concentration of inks and paper dyes, and these materials can interfere with the bio-threat detection. For this reason, alternatives to bulk fluorescence may be useful. The AirSentinel® monitor includes a particle counter/sizer that provides a continuous count of particles being drawn in with air being screened, within pre-determined size ranges. The particle count over a pre-determined window of time (hereafter referred to as the “count” or the “count rate”) within these predetermined ranges provide additional information that enables potential bio-threat agents to be detected. Typically, air that does not contain fine powders will produce count rates that are less than 10,000 particles per second, for particles in the 0.5-10 micron size range. The fluorescence signal from an airborne bio-threat can be enhanced relative to the fluorescence associated with paper dust by contacting the particles with a liquid or gel (or mixing them with a liquid aerosol) that contains stains that fluoresce strongly when bound to nucleic acids or proteins. An impactor or an impinger may be used to contact the particles with a liquid or a gel. Those of ordinary skill in the art will recognize that empirical testing on contaminated and non-contaminated items of mail can be performed to determine useful particle count/particle size parameters for specific substances.

### Tier 1 Bio-Detection Technology

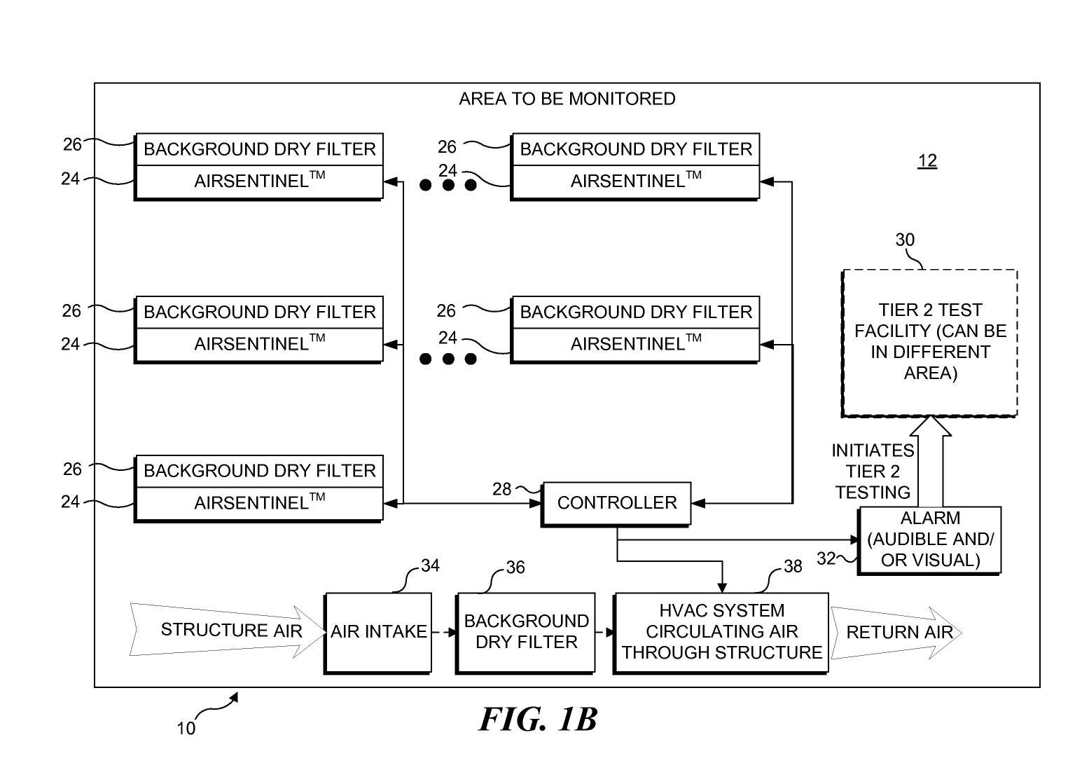

While the following description emphasizes the detection of biological threats, it should be understood that detection of biological airborne threats is intended to be exemplary, and the concepts disclosed herein can also be applied to detecting airborne chemical threats and airborne radiological threats. An AirSentinel® monitor 24a, which is shown in FIG. 2, has an integrated air sampler based on MesoSystems' proprietary rotating impactor technology. The AirSentinel® monitor draws air through a sensor inlet 40, to initially determine if the air is conveying a potential bio-threat agent, and if a potential bio-threat agent is detected, air conveying the potential bio-threat agent is then drawn through a sample inlet 42 to create a sample of the potential bio-threat agent on a substrate disk (not visible in this Figure) for use in carrying out further testing in the Tier 2 procedure, and for use in carrying out any further final confirmation of the Tier 2 results in a clinical laboratory or via a portable verification analytical device . It should be recognized that Tier 1 sampling functions as a trigger, so that if Tier 1 indicates that a potentially harmful agent might be present, additional sampling and analysis is performed in Tier 2, to verify that a harmful agent is actually present, and to attempt to specifically identify the harmful agent.

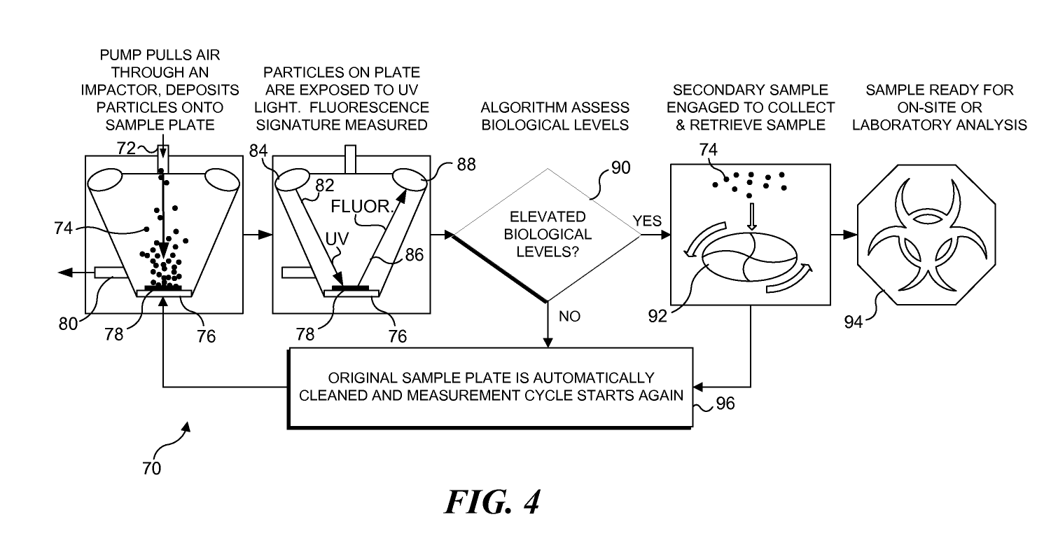

FIG. 4 includes a schematic block diagram 70 that illustrates some of the functions performed within the AirSentinel® monitor to detect a potential bio-threat, and thereafter to collect a sample for further analysis. Air carrying particles 74 is drawn through sample inlet 42 (FIG. 2) and pulled through an air impactor (not shown), exiting through a port 72 for deposition as a spot or sample 78, on a sample plate 76, as shown in FIG. 4. Exhaust air exits through an outlet port 80. Particles deposited as spot or sample 78 are irradiated with an ultraviolet light 82, which is focused by a lens 84. Any fluorescence light 86 emitted by the particles comprising spot or sample 78 is focused by another lens 88 onto a fluorescence light detector (not shown), which produces a corresponding fluorescence signature signal (for example, indicative of the wavelength of the fluorescence light). Based upon the fluorescence signature signal produced by the detector, the logic in the AirSentinel® monitor (or other near-real-time detector) is used in a decision step 90 to determine if there appear to be elevated biological levels corresponding to a potential bio-threat by the particles of the sample just collected on sample plate 76. In addition, the particle count and particle size can be employed in making this determination. If not, sample plate 76 is cleaned in a step 96 to substantially remove the last spot or sample of particles, in preparation for receiving the next spot or sample.

However, if it appears that the particles include a potential bio-threat agent, a secondary sample is collected on a sample plate 92, which can be retrieved for processing in the Tier 2 procedure by trained personnel and optionally, for subsequent final confirmation of the result of that Tier 2 processing by a clinical laboratory (or via a portable verification analytical device), as indicated in block 94. In addition, an alarm signal is produced that is used to initiate Tier 2, by summoning the trained personnel who will be carrying out further testing of the secondary sample collected on sample plate 92. Also, the alarm signal can be employed to evacuate the area proximate the air sensor detecting the potential threat, by alerting personnel of the potential bio-threat hazard with an audio and/or visual alarm, and to control air flow through the area (when possible), to prevent possibly contaminated air from being spread outside the area proximate the air sensor detecting the potential airborne threat.

Tier 1 is designed to operate autonomously (automatically without manual intervention), and if air proximate the air sensor contains a potentially hazardous airborne threat, the Tier 1 air sensors can detect that threat in less than one minute. This rapid response time enables initial minimally disruptive responses (such as preventing air in one part of a structure from moving to other parts of the structure, or preventing personnel from entering or leaving the potentially contaminated area) to be immediately implemented. If the Tier 2 analysis indicates no threat is actually present, such minimally disruptive responses will not have significantly adversely impacted the normal use of the area in which the potential airborne threat was detected. It is expected that ordinary (non-hazardous) air will generate some false alarms periodically, and when that happens, a sample is automatically collected for a Tier 2 analysis, which should quickly determine that the alarm was not justified. Conversely, the Tier 2 analysis can quickly confirm that a real bio-threat agent has been detected by the Tier 1 procedure and then identify the specific bio-threat agent that has been found, so that where a real threat is present, more disruptive but appropriate and necessary responses can be implemented relatively quickly after the threat was initially detected by the first tier.

### Tier 2 Bio-Detection Technology

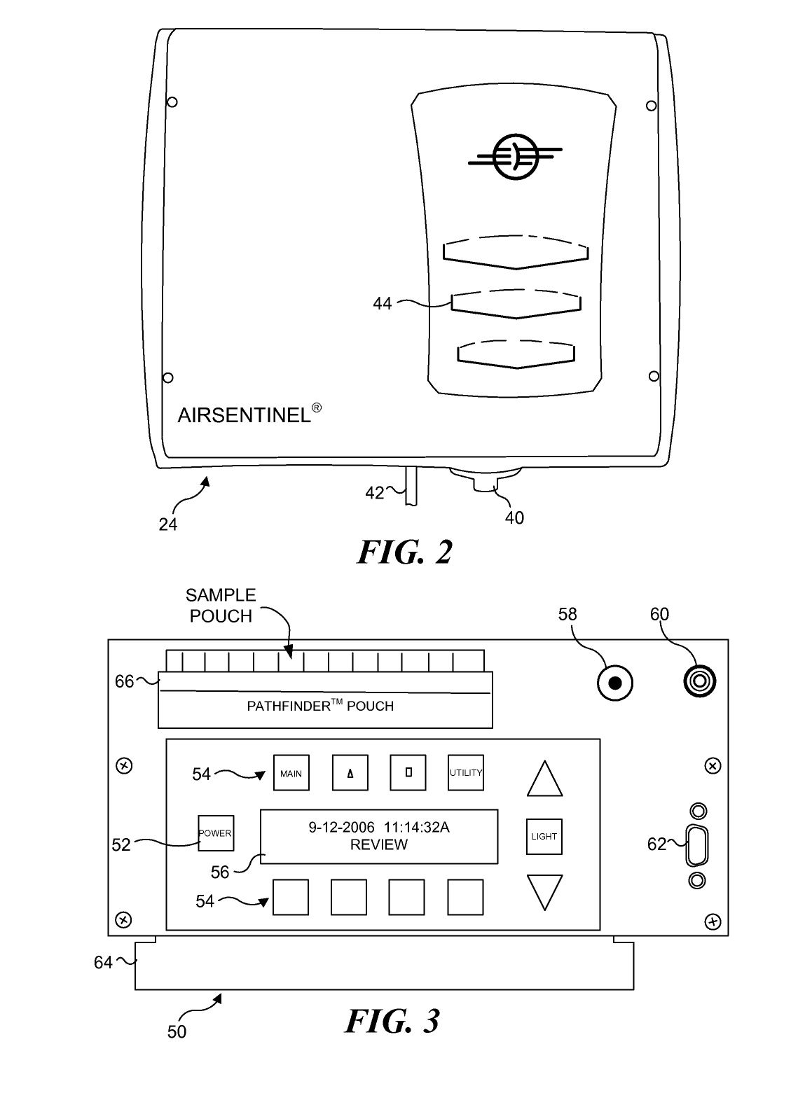

While the following description emphasizes the detection of biological threats, it should be understood that detection of biological airborne threats is intended to be exemplary, and that the concepts disclosed herein can also be applied to detecting airborne chemical threats and airborne radiological threats. The heart of the Tier 2 detection technology configured to verify/identify an airborne biological threat, in one exemplary embodiment, is a Razor™ bio-agent identification system 50, which is shown in FIG. 3. The Razor™ incorporates state-of-the-art PCR “DNA fingerprint” technology currently used by the USPS system. The Razor™ device does not operate continuously, but, when the Tier 1 sensor in AirSentinel® monitor 24a detects a potential bio-threat agent, and in response, generates an alarm signal causing a sample to be collected, that sample is first prepared for analysis by a trained technician and then injected into the Razor™ device. The results of the test are available about 20-30 minutes later. The sample preparation takes about 10 minutes, so a complete Tier 2 test takes approximately 30-40 minutes. The Razor™ system is capable of detecting a very small quantity of a bio-threat agent—i.e., much less than a microgram of powder. It is also able to identify specific threat organisms from among a number of different types of bio-threat agents and is not prone to false positives or false negatives.

The Razor™ system shown in FIG. 3 includes a power switch 52, as well as a plurality of other control buttons 54 on the top panel of the device, disposed around a display screen 56 that displays different messages and indicates the status of the device as it performs different processing functions. Also included on the top of the device are a cover lock 58, which secures a hinged cover 64 (shown in the open position), an external power port 60 (the Razor™ is portable and normally battery operated), and an RS-232 serial data port 62. A plurality of thin-film sample pouches 66 are used as reaction containers for the PCR assay tests that are performed by the Razor™ device.

The drawbacks of Tier 2 are that it does not provide an immediate response, and Tier 2 technologies generally do not operate autonomously. A trained technician is normally required to perform these tests, although as the technology matures, the tests are likely to become more automated and require personnel with less training. Furthermore, as the Tier 2 technology matures, assays that can be completed in much less than 30 minutes are likely to become available. However, Tier 2 does provide an unambiguous test result that is not subject to false alarms, and it can do so within 30 minutes after a Tier 1 alarm is generated. Because the commercially-available Tier 2 tests can be completed in 30 minutes, there is no need to wait for an extended time before verification of the existence of the potential threat is completed by a remote laboratory.

Immunoassay tests and other commercial nucleic acid and protein stain-based tests can be used in connection with PCR, or other genetic fingerprinting assays, as components of Tier 2. Mass spectrometers may all also provide an alternative fingerprinting technology suitable for Tier 2.

### Tier 3 Bio-Detection Technology

While Tier 1 and Tier 2 combine to provide an early warning system, the third tier might be viewed as the “last line of defense.” The third tier includes one background air sampler 26 attached to each Tier 1 air sensor (or disposed proximate to each Tier 1 air sensor) for sampling the air, and background air sampler 36 attached to the air handling system of the structure. At the end of each day, samples of the particulates accumulated (or vapors adsorbed) on the background air samplers are collected, aggregated, and then tested using the Razor™ device, or some other type of manual system for identifying specific bio-threat agents. Note where no air handling system is employed (i.e., in an open area such as a park or open air stadium), the background samplers can simply be disposed proximate to the plurality of Tier 1 detectors. If desired, fewer background samplers than Tier 1 detectors can be employed.

The purpose of Tier 3 is provide a “detect-to-treat” capability similar to the USPS system described above in the Background section, should the Tier 1 system fail to produce an alarm in near-real-time for any reason when a threat is present in the air of the area being monitored. For relatively low concentrations of a bio-threat agent present in ambient air, the background air sampler can collect sufficient amounts of the agent over time, to be more readily detected and identified.

### The CONOPS (Concept for Operations) Approach

During normal operations, ambient air proximate to each of a plurality of Tier 1 air sensors is screened for potential airborne threats. If no alarms are generated, which will normally be the case, no further action is required.

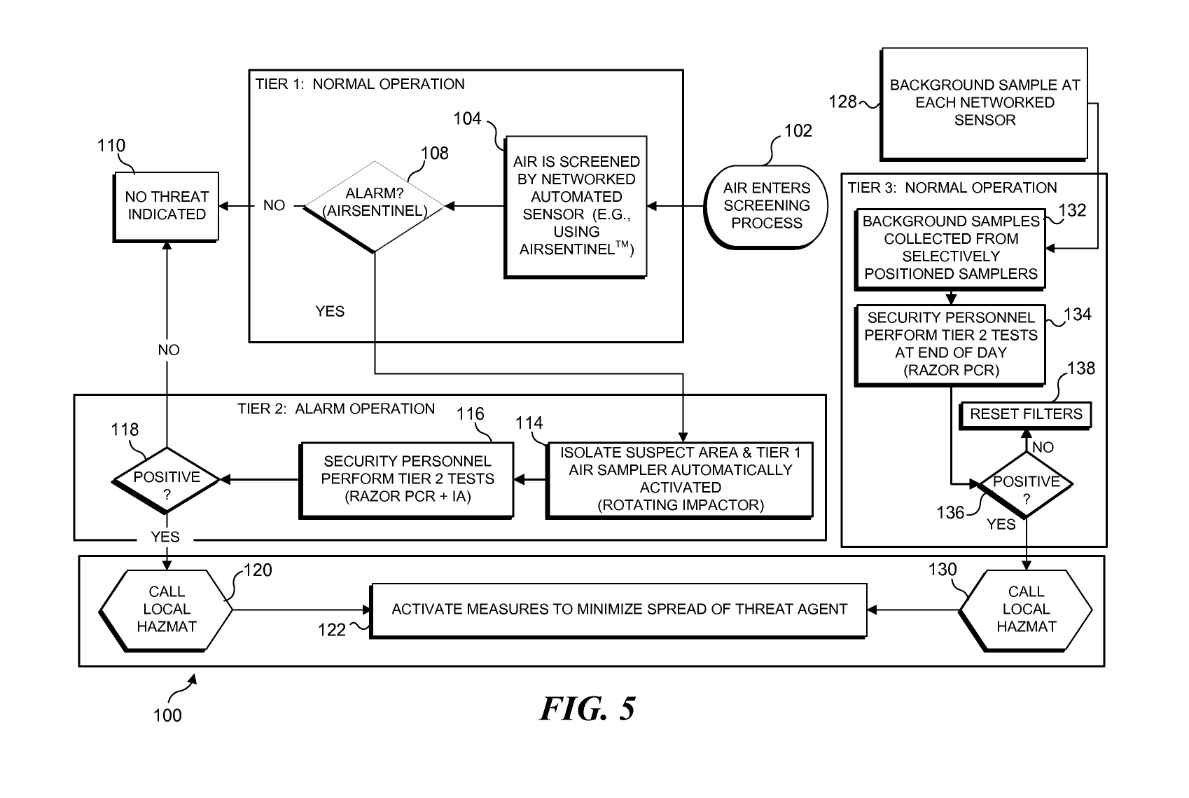

If an alarm is generated, then Tier 2 testing is initiated. In an exemplary embodiment, a pager signal is generated to alert the designated responder (such as a contractor or security officer) of the alarm. Tier 2 testing is performed by trained responder personnel, preferably using portable Tier 2 verification equipment brought to the Tier 1 sensor that detected the potential threat. It should be recognized that in an alternative embodiment, the responder simply collects the sample required for Tier 2 processing and takes the sample to a Tier 2 verification unit. This alternative embodiment may be preferable where it is desirable to perform the Tier 2 analysis out of the public's view (in order to avoid unnecessarily alarming the public, particularly because the Tier 2 testing may indicate that no threat is actually present). The Tier 2 testing can be performed in a designated area out of the public's view, using portable Tier 2 equipment or permanently positioned Tier 2 equipment. It should also be recognized that the Tier 2 testing equipment can be permanently stationed in a vehicle, where the Tier 2 analysis can be performed in private, or permanently station in a testing area within reasonably close proximity to the plurality of Tier 1 sensors (however, performing Tier 2 testing at a site separated from the Tier 1 sensor that detected the potential airborne threat will likely increase the response time required for verification/identification of the potential airborne threat). These operations are shown schematically in a block diagram 100 in FIG. 5. The Figure not only shows the flow of air as it is screened, but also the decision process as alarms are generated if a potential threat is detected. In addition, the Figure indicates where the technology is utilized.

Ambient air enters the Tier 1 screening process at a block 102. In an exemplary embodiment, ambient air is screened using a plurality of Tier 1 air sensors, implemented, for example, using the AirSentinel™ monitor, as indicated in a step 104. If the continuous near-real-time Tier 1 detector, such as the AirSentinel™ monitor, does not detect a potential airborne threat in a decision step 108, no further action (beyond the continuous screening of the ambient air using the Tier 1 sensors) is required, because (assuming the instrument is functioning properly) no potential threat is present (or is present in detectable quantities), as indicated in a step 110. However, if the continuous near-real-time detector, such as AirSentinel™ monitor 24a detects a potential bio-threat (or chemical, or radiological threat), an alarm signal is produced that leads to a step 114 in the Tier 2 procedure. Step 114 provides for isolating the area proximate to the Tier 1 sensor detecting the potential airborne threat (for example, by deactivating HVAC equipment where appropriate, or by preventing people from entering or leaving the immediate area). In addition, in a particularly preferred embodiment, this step provides for automatically collecting a secondary sample using a rotating impactor, as discussed above. Alternatively, but less preferably, the Tier 2 responder can be tasked with collecting the Tier 2 sample. At a step 116, trained responder personnel perform the Tier 2 tests on the sample collected by the near-real-time detector (e.g., the AirSentinel™ monitor) to confirm whether the potential airborne threat agent is actually is a hazardous agent, and if so, to identify the specific airborne threat when possible using the available Tier 2 equipment.

If the potential airborne threat is found to be a harmless substance, in a decision step 118, then no threat is present, as indicated in step 110. Conversely, if the alarm is confirmed and/or a specific airborne threat is identified by the Tier 2 tests, the local hazmat team is immediately called in a step 120. In one embodiment, normal operations proximate to the Tier 1 air sensor that initially detected the potential airborne threat may be stopped, and if desired, personnel in that area can be required to stay in the immediate area to avoid the potential for spread of the airborne contaminant, or instead, can be immediately evacuated. Control of the area proximate to the Tier 1 air sensor that initially detected the potential airborne threat is yielded to the incident commander on the hazmat team as soon as the hazmat team arrives.

However, interrupting normal operations due to a Tier 1 alarm, which may be a false positive, is likely to be unacceptably disruptive. Thus, in an alternative, and more preferred embodiment, no immediate actions that are disruptive to normal operations are taken in response to a Tier 1 alarm, because it is likely that Tier 1 technology will produce more false positives than Tier 2 technology. In such an embodiment, disruptive actions will only be implemented upon a Tier 2 verification that a threat is present. Because the second (and third) tier technologies are so specific, false positives from such tests are likely to be virtually non-existent. Indeed, it is quite possible that a positive Tier 2 test will never occur, because real terrorist events are rare.

Tier 3 testing is performed by security personnel or the designated responder after operations have ended for the day. A step 128 indicates that the system samples air at each Tier 1 sensor over a period of time. In a step 132, background samples taken by the background air samplers (i.e., Tier 3 samplers, preferably implemented as a filter, generally as discussed above) are collected. It should be noted that unless the number of Tier 1 sensors deployed is relatively low, it will likely not be practical to obtain a Tier 3 sample proximate each Tier 1 sensor. In an alternative embodiment, particularly preferred where the sensor network is deployed in a building with an HVAC system, the HVAC system can be segregated into different zones, such that each zone in the HVAC system (or at least those zones that are considered to be most at risk) will have a single Tier 3 sampler. Because samples will be collected from each Tier 3 sampler regularly (daily in at least one embodiment), deploying large numbers of Tier 3 sensors will undesirably increase the number of man hours required to manage the sensor network, thus undesirably increasing operating expenses. Preferably, Tier 3 sensors will be selectively positioned, balancing the desire to obtain representative background samples with the desire to control costs by reducing the number of Tier 3 samplers. Preferably, modest size sensor networks (i.e., for a sensor network deployed in a small office building) will include only one or two Tier 3 samplers, although it should be recognized that any specific number of Tier 3 samplers identified is intended to be exemplary, rather than limiting. In a step 134, trained security personnel perform the Tier 2 tests, for example, using the Razor™ device to carry out PCR testing of the samples. After the Tier 3 test is completed, the background air samplers are recharged (if necessary), in a step 138, and the area is set for normal operations in the morning or following period of operation. In at least one embodiment, if a Tier 3 test returns a positive by identifying a specific airborne threat in any of the background samples, in a decision step 136, the local hazmat team is immediately notified in step 130, and the procedures associated with a Tier 2 positive test are followed, as in step 122. However, it should be noted that the response plan for a positive Tier 3 test might be different for different types of facilities, thus, step 136 as described above is intended to be exemplary, rather than limiting. Note that a positive Tier 3 test (i.e., the collection of a Tier 3 sample and testing the sample using Tier 2 technology) is likely to come too late to protect the building occupants, because the release of the threat agent likely occurred many hours earlier. So, in at least one alternative embodiment, the next step after a positive Tier 3 test or Tier 3 alarm is to collect additional samples, and take the additional samples to an analytical lab for confirmation that there was a “real” attack, and not just the detection of a naturally- occurring trace amount of a threatening agent, or a false alarm from the Tier 2 technology used to test the Tier 3 sample (noting that such false alarms are likely to be rare, but are possible).

Support services that are an integral part of the sensor network disclosed herein include:

Training for the responder personnel on safe operation of the Tier 1, Tier 2, and Tier 3 equipment, including appropriate actions required when a positive test is indicated;

Maintenance and repairs of the equipment; and

Follow-up laboratory analysis on any samples that test negative (if desired).

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for monitoring air in a predefined area including a plurality of spaced apart locations to detect a harmful substance present in the air, comprising the steps of:
(a) automatically sampling the air in the area at a plurality of predefined locations;
(b) automatically evaluating the air sampled at each predefined location to detect a potentially harmful substance in the air proximate to that predefined location, such evaluation being characterized as broadly determining if a potentially harmful substance might be present, rather than identifying a specific harmful substance that might be present;
(c) providing an indication if a potentially harmful substance is detected, the indication specifying at which predefined location the potentially harmful substance might be present; and
(d) in response to the indication of the potentially harmful substance being detected:
(i) collecting a sample of the potentially harmful substance at the predefined location; and
(ii) analyzing the sample of the potentially harmful substance, to confirm the indication and to attempt to identify a specific harmful substance that is present in the air proximate to the predefined location specified by the indication.

2. The method of claim 1, wherein the predefined area comprises at least one element selected from the group consisting essentially of:
(a) a building;
(b) an educational facility;
(c) a facility including indoor and outdoor areas;
(d) a research facility;
(e) an industrial complex;
(f) a military installation;
(g) a transportation facility;
(h) a recreational facility;
(i) an arena;
(j) an entertainment facility;
(k) a food or beverage processing facility;
(l) an agricultural facility;
(m) an indoor facility; and
(n) an outdoor facility.

3. The method of claim 1, wherein the step of collecting the sample of the potentially harmful substance is performed automatically in response to the indication.

4. The method of claim 1, wherein in response to the indication of a potentially harmful substance being detected, further comprising the steps of:
(a) producing an alarm signal that is perceptible by personnel at the predefined location specified by the indication;
(b) producing an alarm signal that is perceptible by personnel tasked with responding to such an indication;
(c) quarantining the predefined location specified by the indication; and
(d) manually collecting the sample of the potentially harmful substance for use in carrying out the assay.

5. The method of claim 1, wherein the step of automatically sampling the air in the predefined area at the plurality of predefined locations comprises the step of continuously sampling the air at least at one of the plurality of predefined locations.

6. The method of claim 1, wherein the step of collecting the sample comprises the step of dispatching a person to the predefined location specified by the indication to collect the sample.

7. The method of claim 1, wherein the step of carrying out the assay of the sample of the potentially harmful substance comprises at least one of the following steps:
(a) analyzing the sample of the potentially harmful substance at the predefined location specified by the indication using a portable analytical device capable of either verifying that a harmful substance is present or specifically identifying at least one harmful substance;
(b) using a portable analytical device capable of either verifying that a harmful substance is present or specifically identifying at least one harmful substance, analyzing the sample of the potentially harmful substance in the predefined area but at a location different than the predefined location specified by the indication, such that the step of analyzing is performed out of public view;
(c) using a non man-portable analytical device capable of either verifying that a harmful substance is present or specifically identifying at least one harmful substance, analyzing the sample of the potentially harmful substance at a location different than the predefined location specified by the indication;
(d) completing the assay within less than about thirty minutes; and
(e) carrying out a rapid assay to determine if the potentially harmful substance is of a biological origin.

8. The method of claim 7, wherein the step of analyzing the sample of the potentially harmful substance at a location different than the predefined location specified by the indication, using a non man-portable analytical device capable of either verifying that a harmful substance is present or specifically identifying at least one harmful substance, comprises at least one step selected from the group consisting essentially of:
(a) analyzing the sample of the potentially harmful substance in a vehicle dispatched to the predefined area to perform the assay;
(b) analyzing the sample of the potentially harmful substance in a secure and private location in the predefined area, such that the assay is performed out of public view;
(c) analyzing the sample of the potentially harmful substance in a secure and private location in an adjacent area, such that the assay is performed out of public view; and
(d) analyzing the sample of the potentially harmful substance at a remote location.

9. The method of claim 1, wherein the step of analyzing the sample comprises at least one of the steps selected from the group consisting of:
(a) performing a stain-based assay to determine if the sample includes an unusually high number of particles of biological origin;
(b) performing a polymerase chain reaction (PCR) amplification and detection assay of the potentially harmful substance configured to identify the presence of a genetic fingerprint of at least one specific harmful substance;
(c) performing an immunoassay test to detect the presence of the potentially harmful substance, where the immunoassay test is selected to identify at least one specific harmful substance; and
(d) performing a nucleic acid assay to identify the presence of the genetic fingerprint of at least one specific harmful substance.

10. The method of claim 1, wherein the step of automatically sampling the air within the predefined area at a plurality of predefined locations comprises at least one step selected from the group consisting of:
(a) continuously sampling the air;
(b) sampling the air in a plurality of rooms of a building;
(c) sampling the air in areas of a structure that are publicly accessible;
(d) sampling the air in a plurality of floors of a building;
(e) sampling the air at entrances that enable people to enter a structure; and
(f) sampling the air at inlets that enable air to enter a structure.

11. The method of claim 1, wherein if the analysis either confirms the indication or identifies a specific harmful substance, then automatically initiating at least one of the following responses:
(a) reducing exposure of personnel to the specific harmful substance;
(b) reducing further contamination of the predefined area, by the specific harmful substance;
(c) filtering the air proximate to the predefined location identified by the indication, to reduce or remove the harmful substance;
(d) adjusting air pressure in the predefined location identified by the indication, to prevent air contaminated by the harmful substance from circulating throughout the predefined area;
(e) isolating the predefined location identified by the indication, to prevent air contaminated by the harmful substance from circulating throughout the predefined area;
(f) where the predefined area is a structure, manipulating air flow in air ducts in the structure to prevent air contaminated by the harmful substance from circulating throughout the structure; and
(g) treating air ducts proximate the predefined location identified by the indication, to neutralize any harmful substance that may be present in the air ducts.

12. The method of claim 1, wherein the potentially harmful substance includes a substance selected from the group consisting of:
(a) bacterial spores;
(b) bacteria;
(c) viruses; and
(d) toxins derived from organisms, either living or once living.

13. The method of claim 1, wherein the step of automatically sampling air at each predefined location comprises the step of collecting a sample of particles carried by the air proximate to the predefined location, and wherein the step of automatically evaluating the air sampled at each predefined location comprises the steps of:
(a) measuring a characteristic of the sample;
(b) as a function of the characteristic of the sample that was measured, automatically determining whether the sample includes a potentially harmful substance; and if so,
(c) producing the indication.

14. The method of claim 13, wherein the step of measuring the characteristic of the sample comprises at least one of the sets of steps selected from the group consisting essentially of:
(a) a first set of steps comprising:
(i) irradiating the sample with light of a specific waveband; and
(ii) sensing a fluorescence light signature produced by the sample after the sample is irradiated with the light of the specific waveband, the fluorescence light signature comprising the characteristic determined for the sample; and
(b) a second set of steps comprising:
(i) combining the particles contained within the sample with a liquid or gel containing a stain that binds preferentially to potential harmful substances, resulting in an enhanced fluorescence characteristic;
(ii) irradiating the sample with light of a specific waveband; and
(iii) sensing a fluorescence light signature produced by the sample after the sample has been combined with the stain and irradiated with the light of the specific waveband, the fluorescence light signature comprising the characteristic determined for the sample.

15. The method of claim 13, wherein the step of measuring the characteristic of the sample comprises the step of determining at least one feature of the sample selected from the group consisting of:
(a) a count of particles comprising the sample that are within one or more pre-determined size ranges;
(b) an infrared light characteristic of the particles comprising the sample; and
(c) a count of the particles exhibiting both a pre-determined shape characteristic and a pre-determined size range.

16. The method of claim 1, wherein the step of automatically evaluating the sampled air from each predefined location comprises at least one of the steps selected from the group consisting essentially of:
(a) using a radiation sensor to measure a level of radiation emitted from the sample, to determine if a potentially hazardous radioactive material is present in the air at the predefined location; and
(b) using a metal oxide-based chemical sensor to determine if a potentially hazardous chemical agent is present in the air at the predefined location.

17. The method of claim 1, further comprising the step of periodically carrying out an assay to detect a background level of a specific harmful substance within the predefined area.

18. The method of claim 17, wherein the step of carrying out the assay to detect the background level of a specific harmful substance within the predefined area comprises at least one of the steps selected from the group consisting of:
(a) performing the assay at each different predefined location, thereby determining a background level for each predefined location;
(b) when the predefined area comprises a structure, performing the assay at a location where air from the structure is discharged into an ambient environment;
(c) performing a nucleic acid amplification and detection assay test of a sample collected from air moving through the predefined area, wherein the nucleic acid amplification and detection assay test is configured to identify at least one specific harmful substance; and
(d) exposing an immunoassay test to the potentially harmful substance, where the immunoassay test is selected to identify at least one specific harmful substance when exposed to the sample collected from air moving through the predefined area.

19. The method of claim 1, further comprising the step of positioning each of a plurality of air sensors proximate to one of each of the predefined locations, each air sensor being configured to:
(a) automatically sample the air proximate to the air sensor,
(b) automatically evaluate the sample;
(c) automatically provide the indication;
(d) be logically connected to a network, such that indications from each air sensor are received at a common monitoring station; and
(e) if the indication has been provided, automatically collect a sample to be assayed to verify the presence of the potentially hazardous substance or to identify a specific hazardous substance.

20. A method for screening air for contamination in a multi-tier approach, comprising the steps of:
(a) in a first tier screening of the air, automatically screening the air at a plurality of locations to detect a potential contaminant that may be conveyed by air, the first tier screening being characterized as broadly determining if a potential contaminant might be present, rather than identifying a specific contaminant;
(b) if a potential contaminant is detected during the first tier screening, collecting a sample of the potential contaminant, and producing a first tier alarm indicating that a potential contaminant has been detected, without specifically identifying a particular contaminant, the first tier alarm identifying the location at which the potential contaminant has been detected; and
(c) initiating a second tier screening of the sample collected during the first tier screening to attempt to identify a contaminant comprising the sample.

21. The method of claim 20, wherein if it is confirmed by the second tier screening that the sample comprises a specific contaminant, initiating a series of predefined steps selected to limit contamination by preventing the specific contaminant from spreading beyond the location at which the specific contaminant was detected

22. A system configured for screening air in a predefined area, to detect contamination by a potential contaminant, in a multi-tier approach, comprising:
(a) a plurality of air contaminant detectors, each air contaminant detector being disposed at a different predefined location associated with the predefined area, each such air contaminant detector being configured to screen air proximate to the predefined location in a first tier screening, to detect a potential contaminant that may be conveyed by the air proximate to the predefined location, the air contaminant detector collecting a sample of any potential contaminant detected and producing an alarm signal when a potential contaminant may have been detected, the air contaminant detector being configured to broadly determine if a potential contaminant might be present, rather than identifying a specific potential contaminant;
(b) a controller coupled to each air contaminant detector, the controller responding to the alarm signal by identifying which of the plurality of air contaminant detectors has detected a potential contaminant, and by initiating a second tier processing of the sample that was collected by each of air contaminant detectors that has detected a potential contaminant; and
(c) a component for use in the second tier processing of the sample collected by each of air contaminant detectors that has detected a potential contaminant, the component being configured to determine if at least one specific contaminant is present in the sample, so that predefined appropriate actions can be initiated to prevent the specific contaminant from spreading beyond the predefined location associated with the air contaminant detector that detected the potential contaminant

23. A method for screening air to detect a harmful substance conveyed by the air, comprising the steps of:
(a) providing a plurality of air sensors, each air sensor being disposed at a different predefined location and being configured to screen air proximate to the predefined location, to detect particles in the air and categorize the detected particles as potentially harmful substances and non-harmful substances, rather than identifying a specific harmful substance;
(b) at each air sensor, automatically sampling air proximate to the predefined location;
(c) at each air sensor, automatically evaluating the air sampled to detect a potentially harmful substance that is carried by the air proximate to the predefined location, to determine if a potentially harmful substance might be present, rather than identifying a specific harmful substance;
(d) at each air sensor, providing an indication if a potentially harmful substance is detected while the air is being sampled, the indication identifying the predefined location corresponding to the indication; and
(e) in response to the indication of a potentially harmful substance being detected, analyzing a sample of the potentially harmful substance, to confirm the indication and to attempt to identify a specific harmful substance that is being conveyed by the air.
